(19) United States
(12) Patent Application Publication
(10) Pub. No.: US 2002/0168400 A1
(43) Pub. Date: Nov. 14, 2002

(54) Collagen/synthetic resin foam wound dressing

(75) Inventor: Manoj K. Jain, Potomac, MD (US)

Correspondence Address: Biocore Medical Technologies, Inc, 11800 Tech Roads, Ste 240, SilverSpring, MD, 20904, US

(73) Assignee: BioCore Medical Technologies, Inc.

(21) Appl. No.: 09/854,029

(22) Filed: May 11, 2001

(51) Int. Cl. A61L 15/00

(52) U.S. Cl. 424/445

(57) ABSTRACT

Improved collagen/synthetic resin foam wound dressings are provided which include a layer of synthetic resin foam (preferably polyurethane foam) together with a layer of freeze-dried collagen applied over and coupled with the resin foam layer. The collagen acts as an exudate absorber, in order to promote wound healing. The dressings are fabricated by first providing a resin foam layer, followed by depositing a collagen dispersion over the foam layer and freeze-drying the dispersion.

### BACKGROUND OF THE INVENTION

1. Field of the Invention

The present invention is broadly concerned with improved collagen/synthetic resin foam wound dressings especially designed for absorbing exudates from wounds; the dressings are preferably made up of a synthetic resin and foam layer (especially medical grade polyurethane foam) with a layer of freeze-dried collagen over the foam layer. The dressings of the invention can be used to good effect in the treatment of many types of wounds from various causes, such as accidental wounds or those resulting from decubitus ulcers.

2. Description of the Prior Art

Collagen is the most prevalent protein in the human body. There are at least thirteen distinct types of collagen, each with its own molecular structure to accomplish specific purposes. For example, certain collagens have self-assembly capabilities that aid and augment human skin tissue healing. Moreover, the use of collagen in wound dressings is advantageous because collagen is very absorbent, it will maintain a moist wound environment and can also be used in combination with topical agents. Moreover, collagen is generally non-adherent to skin and can thus be easily applied and removed from wounds.

A high degree of homology exists between animal and human collagens, so that animal collagen types such as bovine collagen are useful for the treatment of wounds in humans, i.e., the bovine collagens exhibit low immunogenicity when implanted in humans or used as topical dressings on human wounds.

Collagen sponges or foams have long been used as hemostatic agents for tissue repair and as research tools for seeding various cell types to study cell functions in different dimensions. The production of medical devices such as dressings using collagen has typically involved purification of naturally occurring animal collagen to remove substantially all gelatin (to a level of less than 1% by weight), with subsequent freeze-drying and cross linking or other chemical modifications. Freeze-drying substantially alters the characteristics of naturally occurring collagen and the resultant freeze-dried products generally have densities of 1.3 g/ml and above, and altered intrafibral pore sizes.

U.S. Pat. Nos. 4,614,794, 4,320,201, 5,948,429, 4,538,603, 4,404,970, and 4,759,359 describe various types of wound dressings including collagen.

Polyurethane foams have been proposed for a number of medical uses in the prior art. There are numerous advantages in the use of such foams, given that they are semi-permeable and non-adherent to skin. The foams can be manufactured as hydrophilic or hydrophobic as desired, and in their hydrophilic form can provide a moist healing environment while repelling contaminants. See, e.g., U.S. Pat. Nos. 5,565,210, 6,043,406, and EP-A-0171268 which describe the use of various synthetic resin foams for wound treatment.

U.S. Pat. No. 3,800,792 describes a laminated collagen film dressing for burn wounds made up of a relatively thick layer of compressed collagen foam and a layer of non-foamed polyurethane material. The purpose of the dressings described in the '792 patent is to control moisture vapor transmission rates from wounds. To this end, the collagen foam layer has a typical thickness of about 0.3 mm and relatively low absorbence properties, whereas the polyurethane layer is created by dissolving the polyurethane and solvent followed by cast-coating the collagen film with the dissolved polyurethane resin.

### SUMMARY OF THE INVENTION

The present invention provides improved collagen-based wound dressings broadly comprising a layer of synthetic resin foam together with a layer of freeze-dried collagen over and operably coupled with the resin foam layer. A variety of synthetic resin foams may be used in the invention, such as polyurethane, polystyrine, epoxy and polyvinyl chloride films; however, the polyurethane foams are greatly preferred.

The collagen component of the dressings is preferably acid soluble collagen derived from animal sources, and particularly Type I bovine collagen. The collagen layer is of high porosity and is especially designed for absorbing wound exudates.

In manufacturing procedures, a medical-grade synthetic resin foam sheet is covered with a previously prepared aqueous dispersion of collagen; this mixture is then freeze-dried to form the dressings of the invention. Alternately, enhanced dispersion of collagen into the synthetic resin foam layer may be provided by subjecting the poured collagen slurry to pressure conditions prior to freeze-drying. It has been found that the finished dressings in accordance with the invention are especially useful for the treatment of exudating wounds such as bum wounds or those incident to decubitus ulcers.

### DETAILED DESCRIPTION OF THE PREFERRED EMBODIMENTS

The collagen/synthetic resin foam dressings of the invention are produced by first providing a layer of the desired synthetic resin foam, followed by depositing a collagen dispersion over the foam layer and freeze-drying the dispersion. The preferred collagen dispersion is in the form of an aqueous slurry of Type I acid soluble bovine collagen having a collagen content of from about 0.25 to 3% by weight, more preferably from about 0.5 to 1.5% by weight. The pH of the preferred dispersion is from about 1.5 to 5.

A variety of polyurethane foams can be used to good effect in the invention. Generally speaking, such foams should have an average pore size of from about 4-100 pores per inch (ppi), a density of from about 0.15-1.25 g/ml, and a thickness of from about 1 to 5 mm. The well known Rynel medical grade polyurethane foams are especially preferred. Other suitable foams are those disclosed in U.S. Pat. No. 5,571,529, incorporated by reference herein.

In practice, the polyurethane foam layer is placed in a pan or the like, followed by depositing the collagen dispersion over the foam layer by pouring. The composite is then initially frozen to a temperature of from about −5 to −50° C. (more preferably from about −10 to −30° C.) with subsequent freeze-drying at a temperature of below about −10° C. and a reduced pressure of from about 0-5 torr.

The resultant composite dressing presents an absorbent collagen layer together with a synthetic resin foam layer. The collagen layer generally has an intrafibral pore size of from about 25 to 150 microns, more preferably from about 50 to 100 microns. Moreover, the freeze-dried collagen layer exhibits at least about 80% of the helicity of the native collagen used to fabricate the layer, and has a density of from about 0.1-0.6 g/ml. The collagen fraction is essentially a polymer with left-handed helices having an average molecular weight of from about 80,000-120,000 Daltons.

The following example sets forth presently preferred steps for the preparation of a collagen/polyurethane foam wound dressing. It is to be understood, however, that this example is provided by way of illustration and nothing therein should be taken as a limitation upon the overall scope of the invention.

### EXAMPLE

A Type I bovine collagen dough product was obtained having 5.5 ±0.2% by weight collagen, 94.2±0.3% by weight water, a pH of 2.0-3.0, and an HCl content of 0.1-0.3% by weight and a sodium benzoate content of 0.10±0.2% by weight; the dough was free of foreign particles and had a consistent white color. This collagen material was used to form a collagen slurry by mixing 1.5 kg of the collagen dough in 9.0 L of water under a vacuum (>−900 mbar) for three minutes. The vacuum treatment absorbed any air bubbles from the slurry. The slurry was further mixed at room temperature to assure homogeneity, giving a final collagen concentration in the slurry of 0.71% by weight.

A medical grade polyurethane foam sheet of 3 mm thickness was placed in a 17″×13″tray. The foam employed was Rynel 562-B medical grade foam having a nominal thickness of 3±0.76 mm. Approximately 325±25 mL of the collage slurry was poured over the foam. The tray was then shaken to uniformly distribute the collagen slurry on the foam. Next, the tray was frozen to a temperature of −20° C. Thereupon, the tray was freeze dried in a conventional freeze dryer by maintaining the product temperature at −25° C. and a vacuum of 1 torr. After complete freeze drying, the composite was taken out of the freeze dryer and cut into pieces and packaged for later use.

The polyurethane foam and collagen layers in the finished wound dressing do not exhibit a sharp boundary, and the collagen layer may be interspersed within the foam layer for a part or for the entire thickness of the foam layer. Although not always necessary, an adhesive boundary may be applied to the foam dressing for ease of application. For example, conventional materials such as adhesive cloth may be used to support the composite dressing for application thereof to a wound.

The dressings of the invention are simply applied to wounds, with the collagen layer adjacent the wound. In this orientation, the dressings provide excellent absorbence of wound exudates and otherwise promote rapid wound healing.

I claim:

1. A wound dressing comprising:
a layer of synthetic resin foam; and
a layer of freeze-dried collagen over and operatively coupled with the foam layer.

2. The dressing of claim 1, said collagen layer extending at least partially into said foam layer.

3. The dressing of claim 1, said foam layer comprising a foam selected from the group consisting of polyurethane, polystyrene, epoxy and polyvinyl chloride foams.

4. The dressing of claim 3, said foam comprising polyurethane foam.

5. The dressing of claim 3, said foam having an average pore size of from about 4-100 pores per inch (ppi).

6. The dressing of claim 3, said foam having a density of from about 0.5-1.25 g/ml.

7. The dressing of claim 1, said collagen being acid soluble.

8. The dressing of claim 1, said collagen layer having a thickness of from about 0.5 to 4 mm.

9. The dressing of claim 1, said foam layer having a thickness of from about 1 to 5 mm.

10. The dressing of claim 1, said collagen comprising Type I bovine collagen.

11. The dressing of claim 1, said collagen having an intrafibral pore size of from about 25 to 150 microns.

12. The dressing of claim 11, said pore size being from about 50 to 100 microns.

13. The dressing of claim 1, said collagen layers exhibiting at least about 80% of the helicity of the native collagen used to fabricate the layer.

14. The dressing of claim 1, said collagen layer having a density of from about 0.1-0.6 g/ml.

15. The dressing of claim 1, said collagen being a polymer with left-handed helices with an average molecular weight of from about 80,000-120,000.

16. A method of treating a wound, comprising the step of applying the dressing of claim 1 to the wound, with said collagen layer adjacent the wound.

17. A method of fabricating a wound dressing, comprising the steps of:
providing a layer comprising synthetic resin foam;
depositing a collagen dispersion over said foam layer; and
freeze drying said collagen dispersion.

18. The method of claim 17, said dispersion being an aqueous slurry of collagen.

19. The method of claim 18, said slurry having a collagen content of from about 0.25 to 3% by weight.

20. The method of claim 19, said content being from about 0.5 to 1.5% by weight.

21. The method of claim 17, including the step of subjecting said dispersion to a vacuum before said deposition thereof.

22. The method of claim 17, said freeze drying step comprising the steps of initially freezing the collagen dispersion to a temperature of from about −5 to −50° C., and thereafter freeze-drying the frozen collagen.

23. The method of claim 22, said temperature being from about −10 to −30° C.

24. The method of claim 17, said freeze drying step comprising the steps of subjecting the collagen dispersion to a temperature below about −10° C. and a reduced pressure of from about 0-5 torr.

25. The method of claim 17, said collagen layer extending at least partially into said foam layer.

26. The method of claim 17, said foam layer comprising a foam selected from the group consisting of polyurethane, polystyrene, epoxy and polyvinyl chloride foams.

27. The method of claim 26, said foam comprising polyurethane foam.

28. The method of claim 26, said foam having an average cell size of from about 4-100 pores per inch (ppi).

29. The method of claim 26, said foam having a density of from about 0.15-1.25 g/ml.

30. The method of claim 17, said collagen being acid soluble.

31. The method of claim 17, said collagen layer having a thickness of from about 0.5 to 4 mm.

32. The method of claim 17, said foam layer having a thickness of from about 1 to 5 mm.

33. The method of claim 17, said collagen comprising Type I bovine collagen.

34. The method of claim 17, said collagen having an intrafibral pore size of from about 25-150 microns.

35. The method of claim 34, said pore size being from about 50-100 microns.

36. The method of claim 17, said collagen layers exhibiting at least about 80% of the helicity of the native collagen used to fabricate the layer.

37. The method of claim 17, said collagen layer having a density of from about 0.1-0.6 g/ml.

38. The method of claim 17, said collagen being a polymer with left-handed helices with an average molecular weight of from about 80,000-120,000.

39. The method of claim 17, said collagen dispersion having a pH of from about 1.5 to 5.
